(19) United States
(12) Patent Application Publication
(10) Pub. No.: US 2018/0140729 A1
(43) Pub. Date: May 24, 2018

(54) STERILIZATION DEVICE

(71) Applicant: NIKKISO CO., LTD., Tokyo (JP)

(72) Inventors: Hiroki KIUCHI, Hakusan-shi Ishikawa (JP); Tetsumi OCHI, Hakusan-shi Ishikawa (JP)

(21) Appl. No.: 15/873,394

(22) Filed: Jan. 17, 2018

Related U.S. Application Data
(63) Continuation of application No. PCT/JP2016/075191, filed on Aug. 29, 2016.

(30) Foreign Application Priority Data
Sep. 7, 2015 (JP) 2015-176157

(51) Int. Cl. A61L 2/10; A61L 2/00

(52) U.S. Cl. CPC A61L 2/10; A61L 2/0047; A61B 1/121; A61L 2202/24; A61L 2202/122

(57) ABSTRACT

A sterilization device includes a treatment chamber provided with multiple inlet ports and an outlet port, and multiple light sources that irradiate a fluid flowing within the treatment chamber with ultraviolet light. Each of the multiple light sources is disposed so as to emit ultraviolet-light toward a fluid flowing closer to the corresponding inlet port than to the outlet port. The treatment chamber may be of a shape extending in the longitudinal direction from a first end surface toward a second end surface. The multiple light sources may include the first light source disposed on the first end surface and the second light source disposed on the second end surface.

### RELATED APPLICATION

Priority is claimed to Japanese Patent Application No. 2015-176157, filed on Sep. 7, 2015, the entire content of which is incorporated herein by reference.

### BACKGROUND

### 1. Field of the Invention

The present invention relates to a sterilization device, and particularly to a device for irradiating a fluid with ultraviolet light for sterilization.

### 2. Description of the Related Art

Ultraviolet light is known for its sterilization capability, and devices for emitting ultraviolet light are used for sterilization treatment in the field of medical treatment, food processing, or the like. Further, devices for irradiating a fluid, such as water, with ultraviolet light so as to continuously sterilize the fluid are also used. For such sterilization devices, a structure is known in which a turbulence plate or a turbulence generating mechanism is provided midway along a flow path to generate a turbulent-state of the fluid, thereby improving the irradiation efficiency of ultraviolet light for the fluid, for example.

When such a turbulence plate or a turbulence generating mechanism is provided, the flow path structure becomes complicated, causing an increase of necessary parts or the manufacturing cost. Therefore, it is suitable to provide a device with a simpler flow path structure that can improve the irradiation efficiency of ultraviolet light.

### SUMMARY OF THE INVENTION

The present invention has been made in view of such a problem, and an illustrative purpose thereof is to provide a sterilization device with a simple flow path structure and with improved sterilization capability.

A sterilization device according to one embodiment of the present invention includes: a treatment chamber including multiple inlet ports and an outlet port; and multiple light sources that irradiate a fluid flowing within the treatment chamber with ultraviolet light. Each of the multiple light sources is disposed so as to emit ultraviolet light toward a fluid flowing closer to the corresponding inlet, port than to the outlet port.

According to the embodiment, multiple inlet ports are provided in the treatment chamber, thereby providing a place where a turbulent state is generated by inflow to the treatment chamber, at multiple positions within the treatment chamber, Also, since a light source is disposed so as to irradiate, with ultraviolet light, a fluid flowing in the vicinity of an inlet port where a turbulent state is generated, the irradiation efficiency of ultraviolet light for a fluid can be improved. Thus, by combining multiple inlet ports and multiple ultraviolet light sources disposed for the inlet ports respectively, the irradiation efficiency of ultraviolet light for a fluid within the treatment chamber can be improved, thereby also improving sterilization capability.

The treatment chamber may be of a shape extending in the longitudinal direction from a first end surface toward a second end surface. Also, the multiple light sources may include a first light source disposed on the first end surface and a second light source disposed on the second end surface.

The multiple inlet ports may include a first inlet port provided near the first end surface, and a second inlet port provided near the second end surface. Also, the outlet port may be provided between the first inlet port and the second inlet port.

The sterilization device may further include multiple inflow paths connected to the multiple inlet ports respectively and extending in a direction that intersects a longitudinal direction of the treatment chamber.

The multiple inflow paths may extend in a direction perpendicular to a longitudinal direction of the treatment chamber.

The multiple inflow paths may include a first inflow path connected to the first inlet port, and a second inflow path connected to the second inlet port. The first inflow path may extend in a direction that intersects both a longitudinal direction of the treatment chamber and a direction perpendicular to the longitudinal direction so that a fluid flowing through the first inflow path toward the treatment chamber has a velocity component from the first inlet port toward the first end surface. Also, the second inflow path may extend in a direction that intersects both a longitudinal direction of the treatment chamber and a direction perpendicular to the longitudinal direction so that a fluid flowing through the second inflow path toward the treatment chamber has a velocity component from the second inlet port toward the second end surface.

The outlet port may be provided at a position where the distance from the first end surface is equal to the distance from the second end surface, and the first inlet port and the second inlet port may be provided at positions where the distances from the outlet port are the same.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a sectional view that schematically shows a configuration of a sterilization device according to a first embodiment;

FIG. 2 is an external perspective view that schematically shows a flow path structure shown in FIG. 1;

FIG. 3 is a sectional view that schematically shows a configuration of a sterilization device according to a modification;

FIG. 4 is a sectional view that schematically shows a configuration of a sterilization device according to another modification;

FIG. 5 is a sectional view that schematically shows a configuration of a sterilization device according to a second embodiment; and

FIG. 6 is an external perspective view that schematically shows a flow path structure shown in FIG. 5.

### DETAILED DESCRIPTION OF THE INVENTION

The invention will now be described by reference to the preferred embodiments. This does not intend to limit the scope of the present invention, but to exemplify the invention.

Hereinafter, a mode for carrying out the present invention will be described in detail with reference to the drawings. In the description, like reference characters designate like elements, and the same description thereof will be appropriately omitted.

### First Embodiment

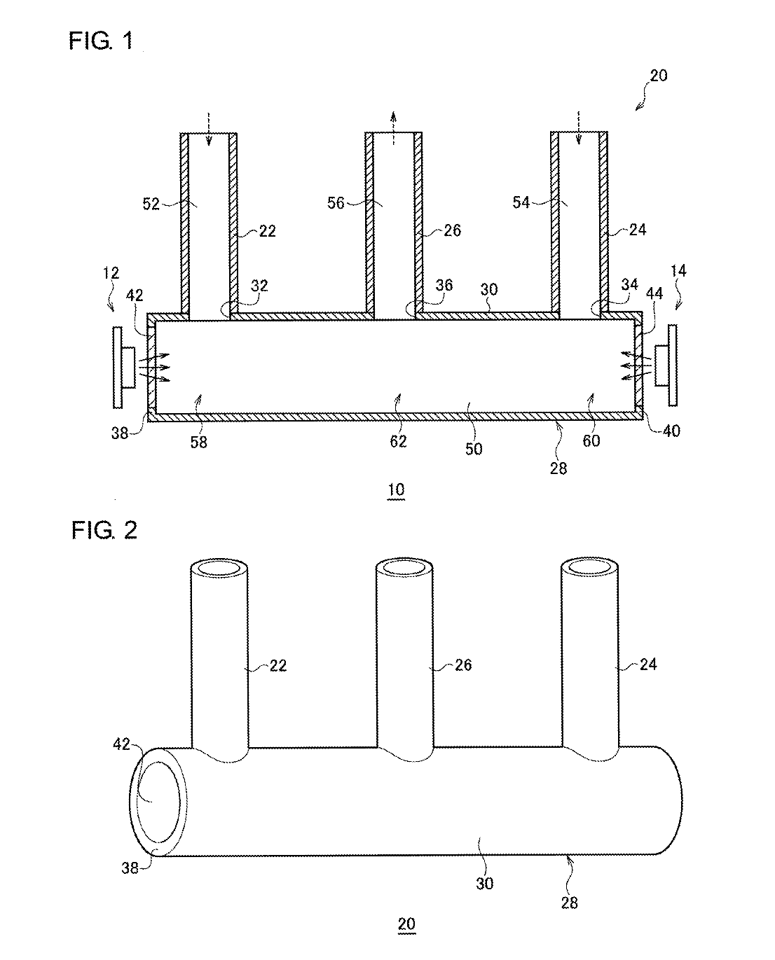

FIG. 1 is a diagram that schematically shows a configuration of a sterilization device 10 according to the embodiment, and FIG. 2 is an external perspective view that schematically shows a flow path structure 20 shown in FIG. 1. The sterilization device 10 comprises multiple light sources (a first light source 12, a second light source 14) and the flow path structure 20. The flow path structure 20 is sectioned into a treatment chamber 50, multiple inflow paths (a first inflow path 52, a second inflow path 54), and an outflow path 56. The sterilization device 10 irradiates a fluid flowing through the first inflow path 52 or second inflow path 54 into the treatment chamber 50 with ultraviolet light emitted by the first light source 12 and second light source 14, and the fluid sterilized by the irradiation of ultraviolet light flows out through the outflow path 56.

The flow path structure 20 includes a first inflow pipe 22, a second inflow pipe 24, an outflow pipe 26, and a casing 28. The flow path structure 20′ is made of a metal material or resin material. The flow path structure 20 is constituted by a member having high durability to ultraviolet light and a high ultraviolet light reflectance. For example, the flow path structure 20 may be made of aluminum (Al) or fluororesin, such as polytetrafluoroethylene (PTFE). Particularly, such a material is desirably used for the inner wall surface of the casing 28, which is directly irradiated with ultraviolet light emitted by the first light source 12 and second light, source 14.

The casing 28 includes a side wall 30, a first-end surface wall 38, and a second end surface wall 40. The side wall 30 has a cylindrical shape as shown in FIG. 2 and extends in the longitudinal direction from the first end surface wall 38 toward the second end surface wall 40. The first end surface wall 38 and the second end surface wall 40 are provided at the both ends of the side wall 30. The side wall 30, first end surface wall 38, and second end surface wall 40 of the casing 28 form the treatment chamber 50. Namely, the treatment chamber 50 is a cylindrical space surrounded by the casing 28 and extending in a longitudinal direction. The treatment chamber 50 is formed to have a cross-sectional area of flow larger than that of the first inflow path 52 or the second inflow path 54.

The first end surface wall 38 is provided with a first window 42 that transmits ultraviolet light emitted by the first light source 12. Also, the second, end surface wall 40 is provided with a second window 44 that transmits ultraviolet light emitted by the second light source 14. Each of the first window 42 and second window 44 is constituted by a member having high ultraviolet light transmittance, such as quartz (SiO₂), sapphire (Al₂O₃), and amorphous fluororesin.

On the side wall 30, a first inlet port 32, a second inlet port 34, and an outlet port 36 are provided. The first inlet port 32 is provided near the first end surface wall 38, and the second inlet port 34 is provided near the second, end surface wall 40. The outlet port 36 is provided at a position between the first inlet port 32 and the second inlet port 34, suitably at the midpoint between the first inlet port 32 and second inlet port 34.

The first inflow pipe 22 is connected to the first inlet port 32, and the second inflow pipe 24 is connected to the second inlet port 34. The first inflow pipe 22 and the second inflow pipe 24 extend in a direction that intersects a longitudinal direction of the casing 28 and, as shown in the figures, extend in a radial direction perpendicular to the longitudinal direction. The first inflow pipe 22 and second inflow pipe 24 may be connected to different fluid sources, or may be connected to branches of a pipe connected to a fluid source in common. The outflow pipe 26 is connected to the outlet port 36 and extends in a radial direction perpendicular to a longitudinal direction of the casing 28, similarly to the inflow pipes.

Each of the first light source 12 and the second light source 14 includes an light emitting diode (LED) that emits ultraviolet light of which the center wavelength or peak wavelength falls within a range of about 200 nm to 350 nm. The first light source 12 and the second light source 14 may suitably include an LED that emits ultraviolet light of which the wavelength falls within a range of about 260 nm to 270 nm, which has high sterilization efficiency. As such an ultraviolet LED, one using aluminum gallium nitride (AlGaH) is known, for example.

The first light source 12 is disposed near the first end surface wall 38 to emit ultraviolet light toward the inside of the treatment chamber 50 through the first window 42. Also, the second light source 14 is disposed near the second end surface wall 40 to emit ultraviolet light toward the inside of the treatment chamber 50 through the second window 44. At least part of the ultraviolet light from the first light source 12 is reflected by the inner surface of the side wall 30 and travels in the longitudinal direction of the treatment chamber 50 toward the second end surface wall 40. Similarly, at least part of the ultraviolet light from the second light source 14 is reflected by the inner surface of the side wall 30 and travels in the longitudinal direction of the treatment chamber 50 toward the first end surface wall 38.

With the configuration set forth above, the sterilization device 10 irradiates and sterilizes a fluid flowing through the first inflow path 52 and the second inflow path 54 into the treatment chamber 50 with ultraviolet light emitted by the first light source 12 and the second light source 14, and the fluid after the treatment flows out through the outflow path 56. At the time, the fluid flowing in through the first inflow path 52 strikes the side wall 30 opposite to the first inlet port 32 or the first end surface wall 38, getting into a turbulent state in a first end part region 58 near the first end surface wall 38. Similarly, the fluid flowing in through the second inflow path 54 strikes the side wall 30 opposite to the second inlet port 34 or the second end surface wall 40, getting into a turbulent state in a second end part region 60 near the second end surface wall 40. The first light source 12 irradiates the fluid in the turbulent state in the first end part region 58 with ultraviolet light, and the second light source 14 irradiates the fluid in the turbulent state in the second end part region 60 with ultraviolet light. The fluid that has flowed into the treatment chamber 50 gradually shifts to a laminar state toward a middle region 62 near the outlet port 36 and then flows out of the sterilization device 10 through the outlet port 36 and the outflow path 56.

According to the present embodiment, since multiple inlet ports are provided, turbulent flows can be generated in multiple regions within the treatment chamber 50. Also, since multiple light sources are provided respectively according to multiple positions where turbulent flows are generated, the fluid in the turbulent states can be irradiated with strong ultraviolet light. Therefore, compared to the case in which a single inlet port is provided or in which a light source is provided near the outlet port where the fluid is in a laminar state, the irradiation efficiency of ultraviolet light for a fluid can be improved.

According to the present embodiment, since a turbulent state is generated by devising the positions of the inlet ports connected to the treatment chamber 50 and the direction of the inflow paths, instead of generating a turbulent flow by providing a turbulence plate or a turbulence generating mechanism within the treatment chamber 50, the flow path structure 20 can be simplified. Therefore, the irradiation efficiency of ultraviolet light for a fluid can be improved, while an increase in necessary parts or the manufacturing cost caused by providing a turbulence generating mechanism can be prevented.

According to the present embodiment, since ultraviolet light is emitted from the both end surfaces of the treatment chamber 50 of a tubular shape, in the longitudinal directions of the treatment chamber 50, ultraviolet light can be provided to the entire inside of the treatment chamber 50. Therefore, besides the end part regions of the treatment chamber 50, which get into a turbulent state, the middle region 62 of the treatment chamber 50 can also be irradiated with ultraviolet light, so that the irradiation efficiency of ultraviolet light for a fluid can be further improved.

According to the present embodiment, since the cross-sectional area of flow of the treatment chamber 50 is made larger than that of each of the multiple inflow paths, the flow rate of a fluid in the treatment chamber 50 can be lowered, so that the residence time of the fluid in the treatment chamber 50 can be increased. With the different cross-sectional areas of flow, a turbulent state can be generated more easily near an inlet port. Such functions can further improve the irradiation efficiency of ultraviolet light for a fluid.

The flow path structure 20 may desirably have a shape that is symmetric with respect to the outflow pipe 26. More specifically, the flow path structure 20 may desirably have a shape symmetric with respect to a plane that is perpendicular to a longitudinal direction of the treatment chamber 50 and that passes through the center position of the outflow pipe 26. In this case, the outlet port 36 is provided at a position where the distance from the first end surface wall 38 is equal to the distance front the second end surface wall 40, and the first inlet port 32 and the second inlet port 34 are provided at positions where the distances from the outlet port 36 are the same. Also, the first inflow pipe 22 and the second inflow pipe 24 are formed to have the same cross-sectional area of flow. By employing such a symmetric structure, the flow of a fluid flowing in through the first inflow pipe 22 and the second inflow pipe 24 can be uniformed, and the fluid after the treatment can be made to smoothly flow out through the outflow pipe 26.

### (First Modification)

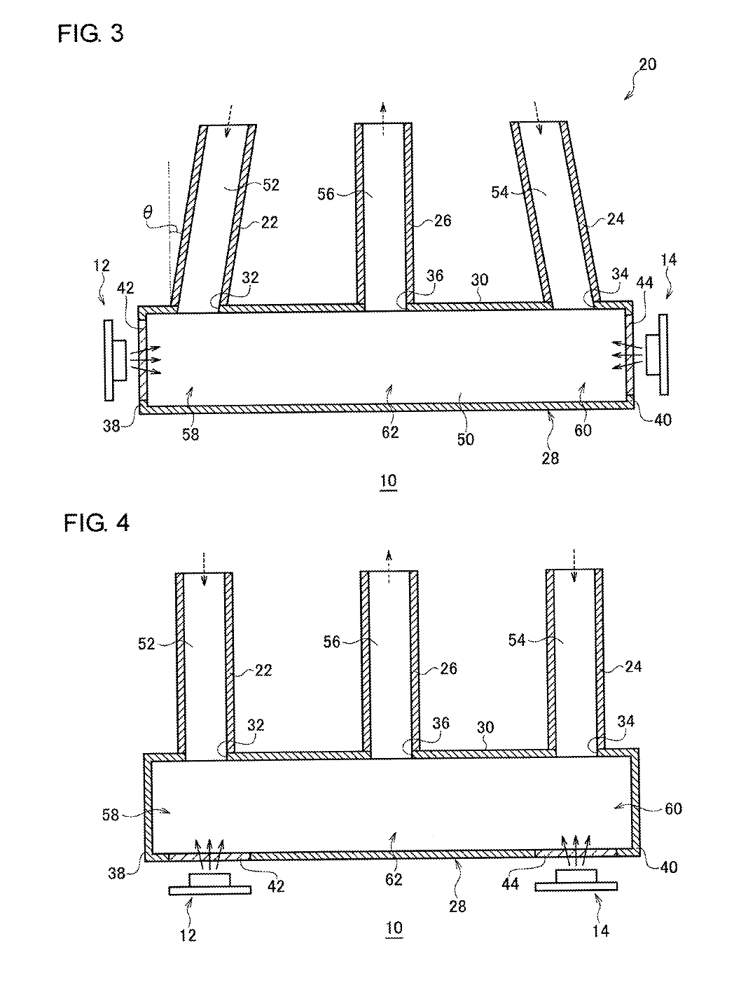

FIG. 3 is a sectional view that schematically shows a configuration of the sterilization device 10 according to a modification. The present modification differs from the aforementioned embodiment in that each of the first inflow path 52 and the second inflow path 54 is provided so as to extend in an oblique direction that intersects both a longitudinal direction and a radial direction of the treatment chamber 50. In the following, the modification will be described mainly for the differences from the aforementioned embodiment.

The first inflow pipe 22 is attached to the first inlet port 32 so as to extend in a direction inclined at an angle θ to a radial direction of the treatment chamber 50. The first inflow pipe 22 is provided so that a fluid flowing through the first inflow path 52 toward the treatment chamber 50 has a velocity component from the first inlet, port 32 toward the first end surface wall 38. Accordingly, the first inflow pipe 22 is provided to be inclined so that the distance from the outflow pipe 26 decreases with increasing distance from the casing 28.

Since the first inflow pipe 22 is provided to be inclined, a fluid flowing through the first inflow path 52 into the treatment chamber 50 is likely to pass near the first end surface wall 38 before flowing toward the outlet port 36. The intensity of the ultraviolet light from the first light source 12 is highest near the first end surface wall 38, so that, by allowing the fluid to pass closer to the first end surface wall 38, the irradiation efficiency of ultraviolet light for the fluid can be further improved.

The angle θ between the radial direction of the treatment chamber 50 and the extending direction of the first inflow pipe 22 may be set arbitrarily, but it may desirably foe set within a range of about 5 to 60 degrees, and more suitably be set within a range of about 10 to 45 degrees, for example. By setting the angle θ to such a value, a turbulent flow is more likely to be generated in the first end part region 58, and the fluid can be made to pass near the first end surface wall 38 before flowing toward the outlet port 36.

As with the first inflow pipe 22, the second inflow pipe 24 is attached to the second inlet port 34 so as to extend in a direction inclined to a radial direction of the treatment chamber 50. The second inflow pipe 24 is provided so that a fluid flowing through the second inflow path 34 toward the treatment chamber 50 has a velocity component from the second inlet port 34 toward the second end surface wall 40. Accordingly, the second inflow pipe 24 is provided to be inclined so that the distance from the outflow pipe 26 decreases with increasing distance from the casing 28.

As with the first inflow pipe 22, since the second inflow pipe 24 is provided to be inclined, a fluid flowing through the second inflow path 54 into the treatment chamber 50 is likely to pass near the second end surface wall 40 before flowing toward the outlet port 36. The intensity of the ultraviolet light from the second light source 14 is highest near the second end surface wall 40, so that, by allowing the fluid to pass closer to the second end surface wall 40, the irradiation efficiency of ultraviolet light for the fluid can be further improved.

The angle between the radial direction of the treatment chamber 50 and the extending direction of the second inflow pipe 24 may be set arbitrarily, but it may desirably be set within a range of about 5 to 60 degrees, and more suitably be set within a range of about 10 to 45 degrees, for example. By setting the angle to such a value, a turbulent flow is more likely to be generated in the second end part region 60, and the fluid can be made to pass near the second end surface wall 40 before flowing toward the outlet port 36. The inclination of the second inflow pipe 24 may desirably be set to the same angle as the inclination of the first inflow pipe 22.

### (Second Modification)

FIG. 4 is a sectional view that schematically shows a configuration of the sterilization device 10 according to another modification. The present modification differs from the aforementioned embodiment in that the first window 42 for transmitting ultraviolet light emitted by the first light source 12 and the second window 44 for transmitting ultraviolet light emitted by the second light source 14 are provided on the side wall 30. In the following, the modification will be described mainly for the differences from the aforementioned embodiment.

The first window 42 is provided on the side wall 30 near the first end surface wall 38, such as at a position opposite to the first inlet port 32. Also, the second window 44 is provided on the side wall 30 near the second end surface wall 40, such as at a position opposite to the second inlet port 34. The first light source 12 is disposed near the first window 42 so as to emit ultraviolet light toward the first end part region 58. Also, the second light source 14 is disposed near the second window 44 so as to emit ultraviolet light toward the second end part region 60.

Also in the present modification, a turbulent flow can be generated in each of the first end part region 58 near the first end surface wall 38 and the second end part region 60 near the second end surface wall 40, and ultraviolet light can be emitted toward a fluid in a turbulent state, so that the irradiation efficiency of ultraviolet light for the fluid can be improved. The positions of the first light source 12 and the second light source 14 may not necessarily be opposite to the first inlet port 32 and the second inlet port 34, and may be positions circumferentially shifted by a given angle from the first inlet, port 32 and the second inlet port 34, respectively.

### Second Embodiment

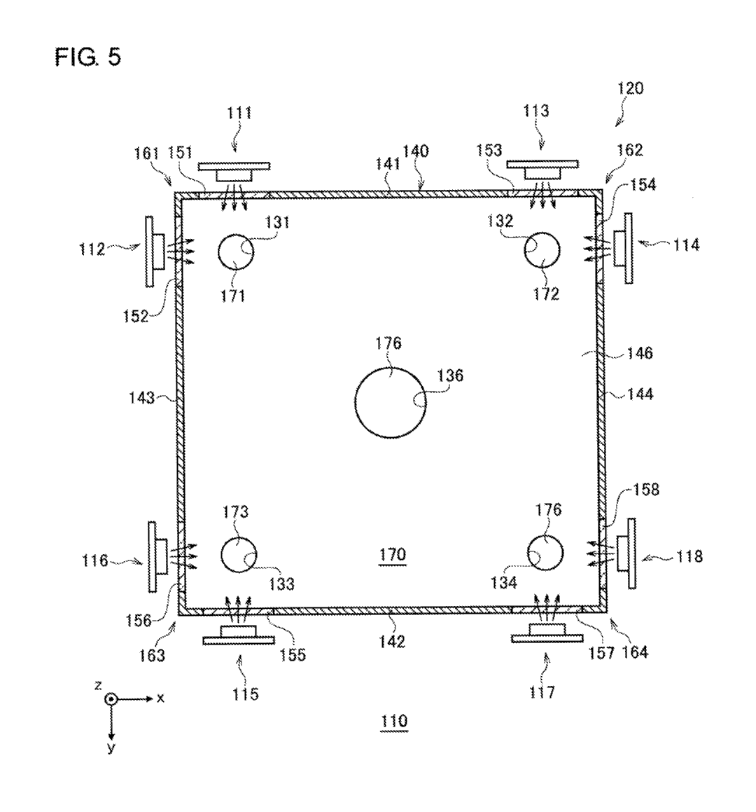

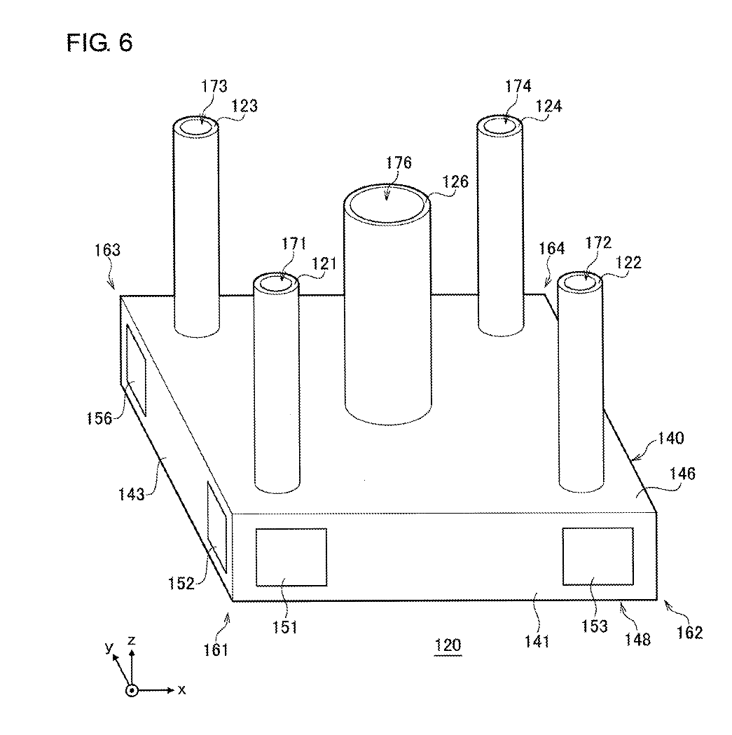

FIG. 5 is a sectional view that schematically shows a configuration of a sterilization device 110 according to the second embodiment, and FIG. 6 is an external perspective view that schematically shows a flow path structure shown in FIG. 5. The sterilization device 110 according to the present embodiment differs from the sterilization device of the aforementioned embodiment in comprising four inlet ports 131-134 as shown in FIG. 5 and four inflow pipes 121-124 as shown in FIG. 6. In the following, description will be given mainly for the differences from the first embodiment.

The sterilization device 110 comprises multiple light sources 111-118 and a flow path structure 120. The flow path structure 120 is sectioned into a treatment chamber 170, multiple inflow paths 171-174, and an outflow path 176. The sterilization device 110 irradiates a fluid flowing through the multiple inflow paths 171-174 into the treatment chamber 170 with ultraviolet light emitted by the multiple light sources 111-118, and the fluid sterilized by the irradiation of ultraviolet light flows out through the outflow path 176.

The flow path structure 120 includes multiple inflow pipes 121-124, an outflow pipe 126, and a casing 140. The casing 140 has a substantially rectangular parallelepiped shape and comprises a first side wall 141, a second side wall 142, a third side wall 143, a fourth side wall 144, an upper surface wall 146, and a lower surface wall 148. In the description of the present embodiment, the directions in which the first side wall 141 and the second side wall 142 face each other are defined as y directions, and the directions in which the third side wall 143 and the fourth side wall 144 face each other are defined as directions. Also, the directions in which the upper surface wall 146 and the lower surface wall 148 face each other are defined as z directions. These directions are defined to assist in understanding of the structure of the sterilization device 110 and do not indicate the directions of the sterilization device 110 during its use.

On the upper surface wall 146, a first inlet port 131, a second inlet port 132, a third inlet port 133, a fourth inlet port 134, and an outlet port 136 are provided. The first inlet port 131 is provided near a first corner 161 at. which the first side wall 141 and the third side wall 143 are in contact with each other, and the second inlet port 132 is provided near a second corner 162 at which the first side wall 141 and the fourth side wall 144 are in contact with each other. Also, the third inlet port 133 is provided near a third corner 163 at which the second side wall 142 and the third side wall 143 are in contact with each other, and the fourth inlet port 134 is provided near a fourth corner 164 at which the second side wall 142 and the fourth side wall 144 are in contact with each other. The outlet port 136 is provided near the center of the upper surface wall 146. Accordingly, the multiple inlet ports 131-134 are provided at opposing corners respectively so as to surround the outlet port 136.

The side walls 141-144 of the casing 140 are provided with multiple windows 151-158. The first window 151 is provided on the first side wall 141 near the first corner 161, and the second window 152 is provided on the third side wall 143 near the first corner 161. The third window 153 is provided on the first side wall 141 near the second corner 162, and the fourth window 154 is provided on the fourth side wall 144 near the second corner 162. The fifth window 155 is provided on the second side wall 142 near the third corner 163, and the sixth window 156 is provided on the third side wall 143 near the third corner 163. The seventh window 157 is provided on the second side wall 142 near the fourth corner 164, and the eighth window 158 is provided on the fourth side wall 144 near the fourth corner 164.

The multiple light sources 111-118 are provided for the multiple windows 151-158, respectively. The first light source 111 is disposed, close to the first window 151 so as to emit ultraviolet light toward a fluid flowing near the first inlet port 131. The second light source 112 is disposed close to the second window 152 so as to emit ultraviolet light toward a fluid flowing near the first inlet port 131. The third light source 113 is disposed close to the third window 153 so as to emit ultraviolet light toward a fluid flowing near the second inlet port 132. The fourth light source 114 is disposed close to the fourth window 154 so as to emit ultraviolet, light toward a fluid flowing near the second inlet port 132. The fifth light source 115 is disposed close to the fifth window 155 so as to emit ultraviolet light toward a fluid flowing near the third inlet port 133. The sixth light source 116 is disposed close to the sixth window 156 so as to emit ultraviolet light toward a fluid flowing near the third inlet port 133. The seventh light source 117 is disposed close to the seventh window 157 so as to emit ultraviolet light toward a fluid flowing near the fourth inlet port 134. The eighth light source 118 is disposed close to the eighth window 158 so as to emit ultraviolet light toward a fluid flowing near the fourth inlet port 134.

Each of the multiple inflow pipes 121-124 and the outflow pipe 126 is attached to the casing 140 so as to extend in a z direction perpendicular to the upper surface wall 146. The first inflow pipe 121 is connected to the first inlet port 131, the second inflow pipe 122 is connected to the second inlet port 132, the third inflow pipe 123 is connected to the third inlet port 133, and the fourth inflow pipe 124 is connected to the fourth inlet port 134. The outflow pipe 126 is connected to the outlet port 136. The multiple inflow pipes 121-124 are configured to have the same cross-sectional area of flow. Meanwhile, the outflow pipe 126 is configured to have a cross-sectional area of flow larger than that of the inflow pipes 121-124.

With the configuration set forth above, in the sterilization device 110, a fluid to be sterilized, is made to flow through the multiple inflow paths 171-174 into the treatment chamber 170, and turbulent flows are generated, near the multiple inlet ports 131-134. Since the multiple light sources 111-118 emit ultraviolet light toward a fluid flowing near the multiple inlet ports 131-134, respectively, the fluid in the turbulent state can be irradiated with strong ultraviolet light. The fluid after the sterilization treatment by irradiation of ultraviolet light then flows out of the sterilization device 110 through the outlet port 136 and the outflow path 176 provided near the center of the treatment chamber 170.

According to the present embodiment, turbulent flows can be generated near the multiple corners 161-164 of the casing 140, and the fluid in the turbulent state can be irradiated with strong ultraviolet light emitted by the multiple light sources 111-118 disposed near the multiple corners 161-164. Therefore, the irradiation efficiency of ultraviolet light for a fluid within the treatment chamber 170 can be improved, similarly to the embodiment described previously.

The flow path structure 120 may desirably have a shape that is symmetric with respect to the outflow pipe 126. For example, the flow path structure 120 may have a shape symmetric with respect to the yz-plane that passes through the center of the outflow pipe 126, in which the vicinity of the first corner 161 corresponds to the vicinity of the second corner 162, and the vicinity of the third corner 163 corresponds to the vicinity of the fourth corner 164. Similarly, the flow path structure 120 may have a shape symmetric with respect to the xz-plane that passes through the center of the outflow pipe 126, in which the vicinity of the first corner 161 corresponds to the vicinity of the third corner 163, and the vicinity of the second corner 162 corresponds to the vicinity of the fourth corner 164. By employing such a symmetric structure for the flow path structure 120, a fluid flowing through the multiple inflow paths 171-174 into the treatment chamber 170 can be made to smoothly flow out through the outflow path 176.

The present invention has been described with reference to the embodiments. It should be understood by those skilled in the art that the invention is not limited to the above-described embodiments and that various modifications could be developed on the basis of various design modifications and such modifications also fall within the scope of the present invention.

The aforementioned first embodiment describes the case where the treatment chamber 50 has a cylindrical shape. In a further modification, the treatment chamber may have a prism shape, and the shape of the both end surfaces facing in the longitudinal directions may be a triangle, a quadrangle, a hexagon, or an octagon.

The aforementioned first embodiment describes the case where multiple light sources are disposed on the end surfaces of the treatment chamber, and the aforementioned modification describes the case where multiple light sources are disposed on the side wall. In a further modification, multiple light sources may be disposed on both the end surfaces and side wall of the treatment chamber.

The aforementioned second embodiment describes the case where the treatment chamber 170 has a rectangular parallelepiped shape. In a further modification, the treatment chamber may have a cylindrical shape, or a prism shape in which the shape of the upper surface wall and the lower surface wall is a triangle, a hexagon, or an octagon. Also, although the embodiment describes the case where the number of the multiple inflow paths connected to the treatment chamber 170 is four, the number is not limited thereto and may be three, or five or more. In this case, it is desirable to form the flow path structure so that the multiple inflow paths are arranged to be symmetric with respect to the outflow path.

The sterilization devices according to the aforementioned embodiments are described as devices for performing sterilization treatment by irradiating a fluid with ultraviolet light. In a modification, the sterilization devices may be used for purification treatment for decomposing an organic substance included in a fluid by irradiation of ultraviolet light.

It should be understood that the invention is not limited to the above-described embodiment, but may be modified into various forms on the basis of the spirit of the invention. Additionally, the modifications are included in the scope of the invention.

What is claimed is:

1. A sterilization device, comprising:
a treatment chamber including a plurality of inlet ports and an outlet port; and
a plurality of light sources that irradiate a fluid flowing within the treatment chamber with ultraviolet light, wherein
the plurality of light sources are disposed at positions corresponding to the plurality of inlet ports respectively so that a fluid flowing near the corresponding inlet port of each of the light sources is irradiated with stronger ultraviolet light than a fluid flowing near the outlet port.

2. The sterilization device of claim 1, wherein:
the treatment chamber has a shape extending in the longitudinal direction from a first end surface toward a second end surface; and
the plurality of light sources include a first light source provided on the first end surface and a second light-source provided on the second end surface.

3. The sterilization device of claim 2, wherein:
the plurality of inlet ports include a first inlet port provided near the first end surface and a second inlet port provided near the second end surface; and
the outlet port is provided between the first inlet port and the second inlet port.

4. The sterilization device of claim 3, further comprising a plurality of inflow paths connected to the plurality of inlet ports respectively and extending in a direction that intersects a longitudinal direction of the treatment chamber.

5. The sterilization device of claim 4, wherein the plurality of inflow paths extend in a direction perpendicular to a longitudinal direction of the treatment chamber.

6. The sterilization device of claim 4, wherein:
the plurality of inflow paths include a first inflow path connected to the first inlet port and a second inflow path connected to the second inlet port;
the first inflow path extends in a direction that intersects both a longitudinal direction of the treatment chamber and a direction perpendicular to the longitudinal direction so that a fluid flowing through the first inflow path toward the treatment chamber has a velocity component from the first inlet port toward the first end surface; and
the second inflow path extends in a direction that intersects both a longitudinal direction of the treatment chamber and a direction perpendicular to the longitudinal direction so that a fluid flowing through the second inflow path toward the treatment chamber has a velocity component from the second inlet port toward the second end surface.

7. The sterilization device of claim 3, wherein;
the outlet port is provided at a position where the distance from the first end surface is equal to the distance from the second end surface; and
the first inlet port and the second inlet port are provided at positions where the distances from the outlet port are the same.

8. The sterilization device of claim 1, wherein each of the plurality of light sources includes an LED that emits ultraviolet light.

9. The sterilization device of claim 1, wherein the inner surface of the treatment chamber contains aluminum.

10. The sterilization device of claim 1, wherein the inner surface of the treatment chamber contains fluororesin.

11. The sterilization device of claim 1, wherein a turbulent flow is generated near each of the plurality of inlet ports.
